(19) United States
(12) Patent Application Publication
(10) Pub. No.: US 2014/0309648 A1
(43) Pub. Date: Oct. 16, 2014

(54) REAM SLEEVE AND METHOD FOR USE THEREWITH

(71) Applicant: EPIX Orthopaedics, Inc., Los Altos, CA (US)

(72) Inventor: Amir M. MATITYAHU, Los Altos, CA (US)

(21) Appl. No.: 14/209,743

(22) Filed: Mar. 13, 2014

Related U.S. Application Data
(60) Provisional application No. 61/801,319, filed on Mar. 15, 2013.

(51) Int. Cl. A61B 17/17

(52) U.S. Cl. CPC A61B 17/1717
USPC 606/96

(57) ABSTRACT

A sleeve to create a bore in a bone comprising a tube adapted to receive a reamer. A protuberance is provided on a side at the distal end of the tube and extends distally of the tube, A hand-holdable element is joined to the proximal end of the tube. The protuberance can be inserted into an opening in the bone to facilitate alignment of the reamer with the bone.

### CROSS REFERENCE TO RELATED APPLICATION

This application claims priority to U.S. provisional patent application Ser. No. 61/801,319 filed Mar. 15, 2013, the entire content of which is incorporated herein by this reference.

### FIELD OF THE INVENTION

The present invention relates to tools for use in creating a bore in a bone and, more particularly, to a sleeve for use with a reamer to create a bore in a bone.

### BACKGROUND OF THE INVENTION

Intramedullary rods or nails have been used to treat femoral, tibial, humeral and other bone fractures. Moreover, they have been used for fusions and repairs of unhealed or crooked bones. Femur, tibia, and humerus fractures, for example, have been treated with rod assemblies that for example are inserted into the canal to coapt the fractured parts. Peritrochanteric fractures of the femur, for example, have been treated with femoral rod assemblies that for example are inserted into the femoral canal to coapt the femur fractured parts. One or two angled cross-nails or locking screws are inserted through the bone at the proximal and distal ends of the intramedullary rod. Rods have been provided that permit the angle of the proximal screw relative to the rod to be adjusted in situ.

Tools have been provided for reaming a bone in a mammalian body to create a bore or canal into which such a rod is inserted. However, difficulties can arise the bore is not properly placed in the bone, or the bone must be otherwise reamed again to create a new bore nearby the first bore.

Tools have additionally been provided for reaming bores or canals in other bones of a mammalian body for other purposes.

There is a need for an improved tool for facilitating the creation of correctly-placed bores in a bone for use with a device for implantation into the bone or otherwise, or for properly placing bores in bones of a mammalian body

### BRIEF DESCRIPTION OF THE DRAWINGS

The drawings described herein are for illustration purposes only and are not intended to limit the scope of the present disclosure in any way.

FIG. 1 is an elevational view of a ream sleeve of the present invention.

FIG. 2 is a first side view of the ream sleeve of FIG. 1 taken along the line 2-2 of FIG. 1.

FIG. 3 is a second side view of the ream sleeve of FIG. 1 taken along the line 3-3 of FIG. 1.

FIG. 4 is a bottom plan view of the ream sleeve of FIG. 1 taken along the line 4-4 of FIG. 1.

FIG. 5 is an enlarged view of a portion of the offset ream sleeve of FIG. 1 take along the line 5-5 of FIG. 4.

FIG. 6 is a side elevational view of the ream sleeve of FIG. 1 in one method of use with a fractured bone.

FIG. 7 is an isometric view of the ream sleeve of FIG. 1 in another method of use with a fractured bone.

FIG. 8 is an enlarged view of a portion of FIG. 7 taken along the line 8-8 of FIG. 7.

FIG. 9 is an enlarged view, similar to FIG. 8, of the ream sleeve of FIG. 2 in a subsequent step of the method of FIG. 7.

### DETAILED DESCRIPTION OF THE INVENTION

In general, tool is provided for creating bores in bones of a mammalian body for treating fractures, nonunions or malunions of the bone. Such bores can be used for a variety of purposes, include the insertion of an implantable device in the bone. One such implantable device is an intramedullary or rod or nail, which can be of any suitable type and in one embodiment can be of the type disclosed in U.S. Publication No. 2009/0048600 published Feb. 19, 2009, the entire contents of which are incorporated herein by this reference. Such rods or nails may also be used for fusion across bones such as the femur-tibia and calcaneus-talus-tibia.

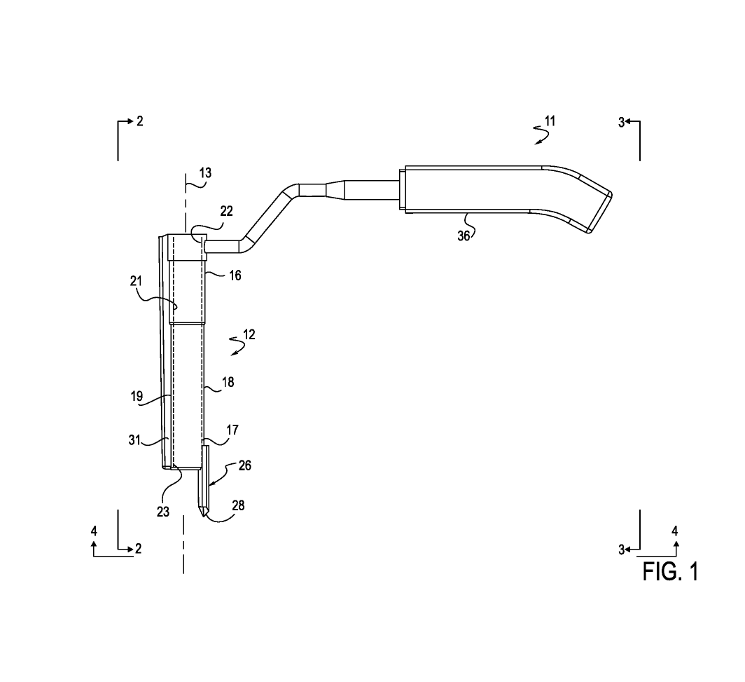

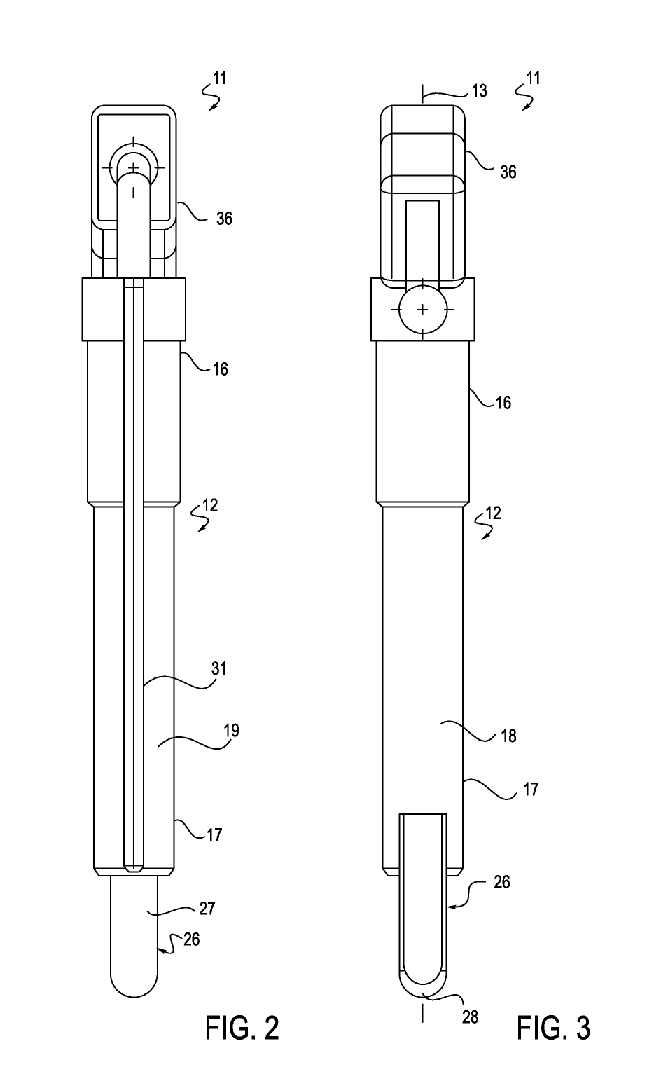

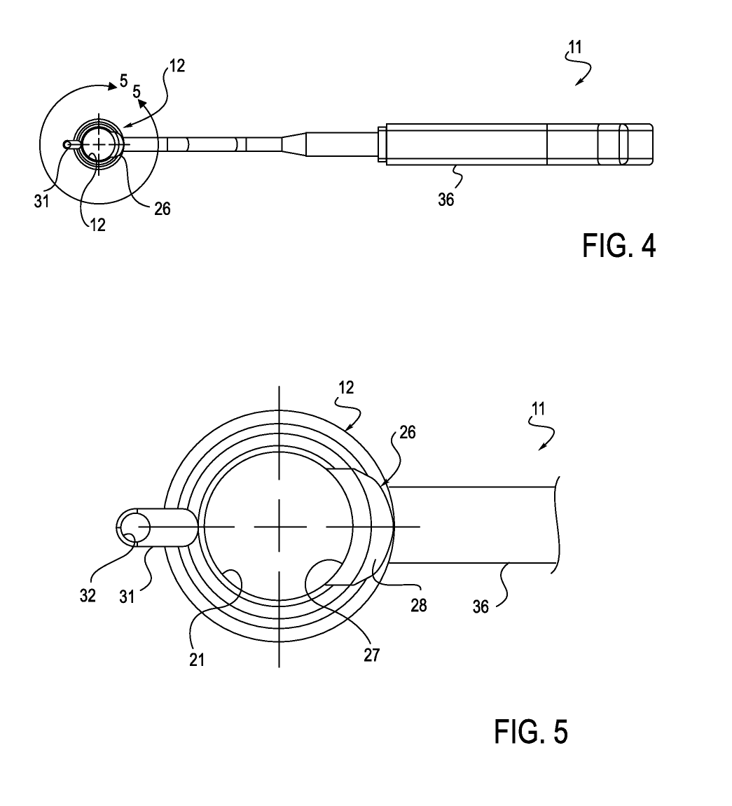

Tool or ream sleeve 11 can be of any suitable type and in one embodiment has a tube or main tube 12 made from any suitable material such as stainless steel. Tube 12 extends along and in one embodiment is centered on a longitudinal axis 13 and has a proximal end 16 and a distal end 17 and a first side 18 and an opposite second or additional side 19 extending be the proximal and distal ends 16, 17 (see FIGS. 1-5). A central bore or passageway 21 extends longitudinally through the tube 12 from a proximal opening 22 and a distal opening 23.

An extension or extension element, protuberance, tab or protrusion 26 is provided on distal end 17 of the first side 18 of tube 12, and extends longitudinally along a portion of the tube and distally of the tube. In this manner, the protrusion 26 extends from a lateral side of the main tube 12. Protrusion or tab 26 is formed with an arcuate inner surface 27, which in one embodiment has a radius approximating the radius of bore 21 of the tube 12, and a tapered end or distal end 28. In one embodiment, protuberance or tab 26 is lateral of the larger or main tube 12.

An optional guide tube 31 can be provided on another side of the tube 12, and in one embodiment is provided on the second side 19 of the tube. In one embodiment, the guide tube 31 is medial of the larger or main tube 12. Guide tube 31 extends longitudinally along the side 19 over at least a portion of the length of the tube and in one embodiment extends along the entire length of the tube. The guide tube 31 is provide with an internal bore or passageway 32 extending longitudinally therethrough and in one embodiment extending parallel to the longitudinal axis 13 of the main tube 12. Passageway 32 in the guide tube 31 can be referred to as an offset channel or offset pin channel.

A hand-holdable element 36 can be joined to the main tube 12 and in one embodiment is joined to the proximal end 16 of the tube 12. Although the hand-holdable element can be of any suitable type, in one embodiment the hand-holdable element is a suitable handle 36 which is configured or adapted for gripping by a human hand.

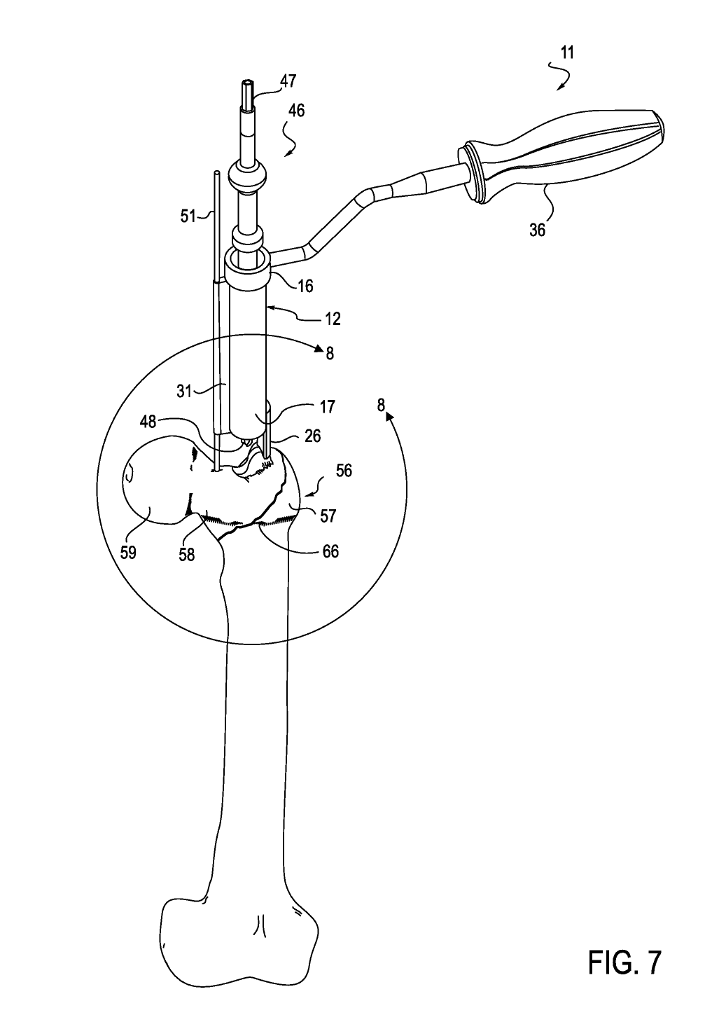

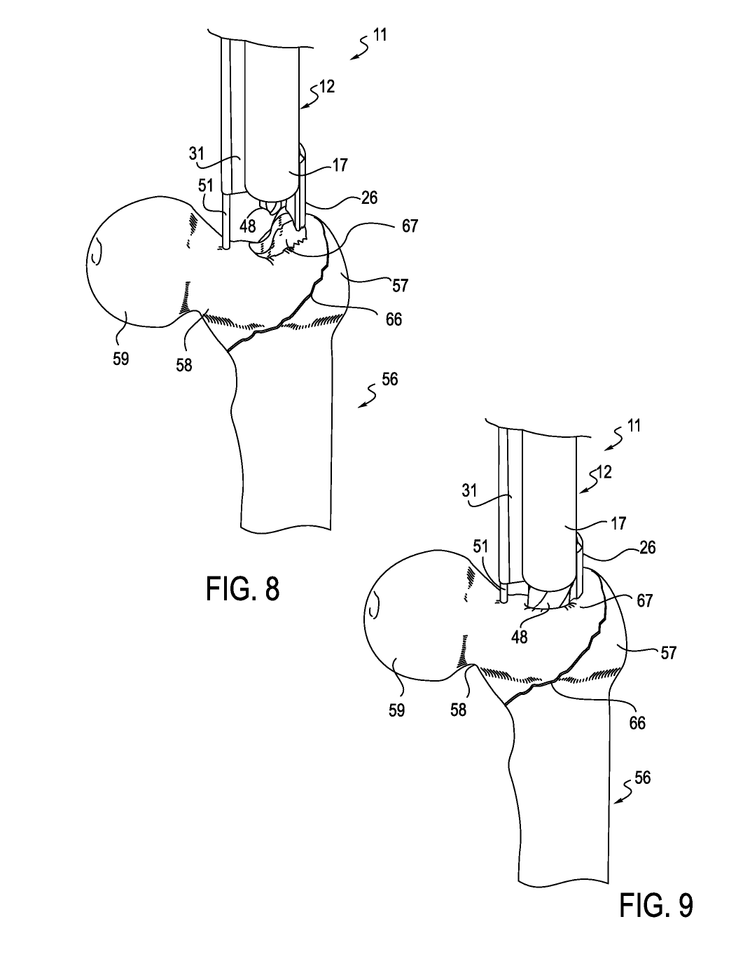

The tool or ream sleeve 11 can be used with a conventional ream tool which can be of any suitable type. In one embodiment, a ream tool or reamer 46 can be provided and have a proximal end 47 and a distal end 48 (see FIGS. 7-9). The proximal end 47 of the elongate ream tool can include a shank 47 suitable for being non-rotatably grasped by a chuck of a suitable drilling device or drill (not shown). The distal end of the reamer can include a bit 48 having a plurality of cutting edges suitable for creating a bore in a bone of a mammalian body. Reamer 46 is suitable sized for relatively snug slidable and rotatable placement in bore 21 of the main tube 12. Main tube 12 is adapted or configured to receive the reamer 46.

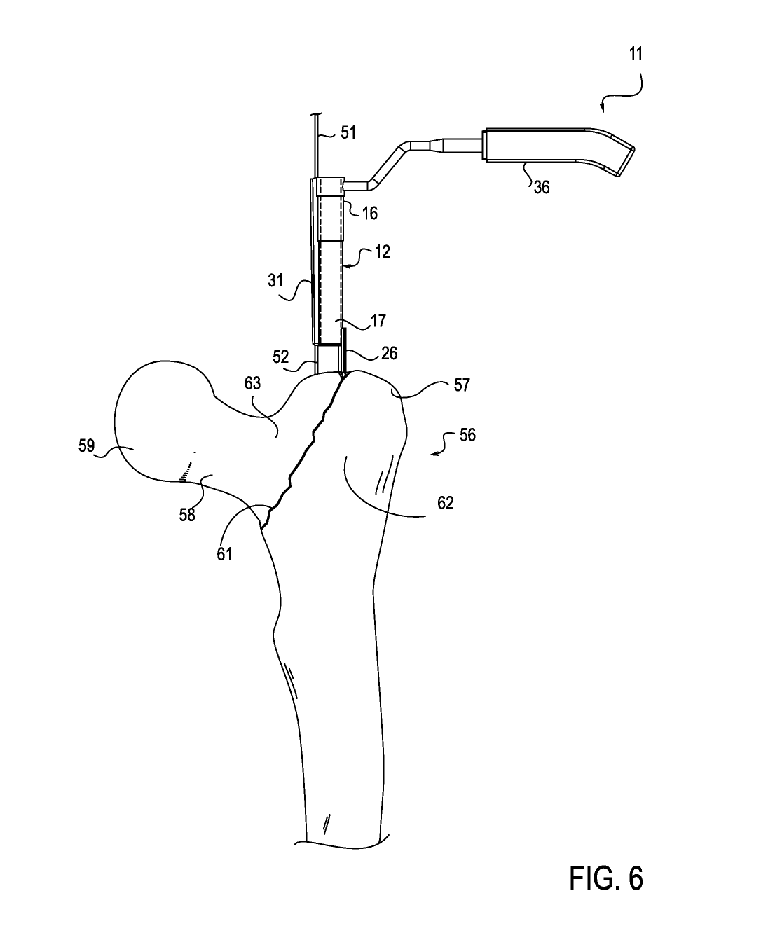

A suitable guide pin 51 can be used with sleeve 11 for extending through the passageway 32 of guide tube 31 (see FIGS. 6-9). The guide pin 51 can be of any suitable type and in one embodiment can be an elongate flexible element having an external diameter smaller than the internal diameter of guide passageway 32. In one embodiment, the guide pin 51 has a sharpened distal tip 51 which can at least slightly penetrate the bone when engaging the bone so as to serve as a registration or alignment pin for positioning the sleeve 11 relative to the bone.

In one method of operation and use of the ream sleeve of the present invention, sleeve 11 can be used for positioning reamer 46 with respect to a bone of a mammalian body for creating a bore or canal in the bone, for example adjacent a fracture in the bone. In one suitable such method, the bone is a femur 56 having a greater trochanter 57 and neck 58 extending to a head 59 at one end of the bone. A fracture 61 extends from the greater trochanter 57 downwardly into the bone through the base of the neck 58, and is sometimes referred to as a simple fracture of the subtrochanteric region. Such fracture can also be referred to as a hip fracture or simple hip fracture. The lateral side of the fracture is identified by reference numeral 62 in FIG. 6, and the medial side of the fracture is identified by reference numeral 63 in FIG. 6. When it is desired to create a bore in the greater trochanter 57 adjacent to fracture 61, for example in the medial side 63 of the greater trochanter 57, protrusion 26 of the ream sleeve 11 can be placed into the fracture 61 so as to abut the medial side 63 of the bone, as illustrated in FIG. 6. Guide pin 51 can be inserted into the passageway 32 of the guide tube 31 and extend distally of the guide tube 31 and main tube 12 so that the sharpened distal tip 52 of the guide pin 51 engages the medial side 63 of the bone in a position spaced apart from the protrusion 26 and the fracture 61. Such engagement of the bone by protrusion 26 and guide pin 51 can serve to align the tube 12 relative to the tube 12 and retain or fix the tube 12 in this position relative to the femur 56 as reamer 46 is introduced through bore 21 of the tube 12 and the tapered distal end of the bit 48 bores into the bone to create or ream a bore in the desired location of the femur and in the desired direction. In such operation of sleeve 11, fracture 61 can be referred to as an opening in the bone for receiving protrusion 26, which can engage or be urged again the bone at a side of the opening to desirably register the main tube 12 and thus reamer 46 relative to the bone.

Ream sleeve 11 desirably retains the reamer 46 in position relative to the fracture 61, and inhibits the lateral and medial sides of the fracture 62, 63 from being pushed apart during creation of the bore in the femur 56. The guide tube 31 and the guide pin 51 slidably carried therein can serve to fixate the medial aspect of the fracture so that when the reamer 46 rotatable and distally extends through tube 12 the reamer will ream more medial than it would have reamed without the offset aspect of sleeve 11.

In another method of operation and use of ream sleeve of the present invention, sleeve can be used for positioning reamer 46 with respect to a bone of mammalian body for creating a bore or canal in a bone adjacent to an existing bore in the bone. In one such operation, illustrated in FIGS. 7-9 with respect to a femur have a fracture 66 and a first bore 67 extending from the greater trochanter 57 in an effort to repair the fracture 66. However a further bore in the femur 56 is required to properly position a nail in the femur to repair the fracture 66. In one method of creating such a second bore 68 in the femur, protrusion 26 extending distally of the main tube 12 can be inserted or placed in the first bore 67, for example as shown in FIGS. 7-9 against the lateral side of the first bore 67, the guide pin 51 can engage the femur 56 medially of the first bore 67. Such engagement of the bone by protrusion 26 and guide pin 51 can serve to align the tube 12 relative to the tube 12 and retain or fix the tube 12 in this position relative to the femur 56 as reamer 46 is introduced through bore 21 of the tube 12 and the tapered distal end of the bit 48 bores into the bone to create or ream the second bore 68 medially of the first bore 67, as shown in FIG. 9. In the illustrated embodiment of FIGS. 7-9, the second bore 68 intersects the first bore 67. In such operation of sleeve 11, the first bore 67 can be referred to as an opening in the bone for receiving protrusion 26, which can engage or be urged again the bone at a side of the first bore 67 to desirably register the main tube 12 and thus reamer 46 relative to the bone.

Protrusion 26 can be of any suitable width, and in one embodiment has a width of approximately four millimeters. It is appreciated, however, that the width and length of the protrusion 26 can be suitably sized to obtain the desired amount of offset with the ream sleeve of the present invention. Main tube 12 can be suitably sized in length and width as required for the intended operation and patient. Further, guide tube 31 can be suitably sized and suitably offset relative to central axis 13 of the ream sleeve 11 for the intended operation and patient.

It is appreciated that the ream sleeve of the present invention can be used for a variety of applications, including repair of a bony canal created along a fracture that needs to be moved medially or laterally, repair of canals or bores that are not of sufficient diameter or in the wrong location, repair of a canal that is offset laterally due to the reamer being pushed laterally by muscle tissue, and repair of a canal that was reamed in an incorrect location due to a guidewire being placed incorrectly.

I claim:

1. A sleeve for use with a reamer to create a bore in a bone, comprising a tube adapted to receive the reamer, the tube having a proximal end and a distal end and a side extending between the proximal and distal ends, a protuberance provided on the side at the distal end and extending distally of the tube and a hand-holdable element joined to the tube at the proximal end whereby the protuberance can be inserted into an opening in the bone to facilitate alignment of the reamer with the bone.

2. The sleeve of claim 1 for use with a guide pin, wherein the tube has an additional side extending between the proximal and distal ends, a guide tube extending longitudinally on the additional side of the tube and adapted to slidably receive the guide pin whereby the guide pin can be slidably introduced through the guide tube to engage the bone in a position spaced from the protuberance for facilitating alignment of the reamer with the bone.

3. The sleeve of claim 2, wherein the additional side is opposite the first-named side.

4. The sleeve of claim 1, wherein the hand-holdable element is a handle.

5. The sleeve of claim 1, wherein the protuberance has a tapered end.

6. A method for use with a reamer create a bore in a bone having an opening therein, comprising providing a tool having a tube for receiving the reamer, the tube having a proximal end and a distal end and a side extending between the proximal and distal ends, a protuberance provided on the side at the distal end and extending distally of the tube, approaching the bone with the distal end of the main tube, extending the protuberance into the opening of the bone so that the protuberance engages the bone in the opening to align the tube relative to the bone, extending the reamer through the main tube and distally of the distal end for engaging the bone and creating a bore in the bone.

7. The method of claim 6, wherein the opening is a bore in the bone.

8. The method of claim 6, wherein the opening is a fracture in the bone.

9. The method of claim 6, wherein the tube has an additional side extending between the proximal and distal ends and a guide tube extending longitudinally on the additional side of the tube, further comprising the step of introducing a guide pin through the guide tube to engage the bone in a position spaced from the protuberance for facilitating alignment of the reamer with the bone.
